SEQUENCE LISTING (6 sequences)

SEQ ID NO: 1 - DNA, 19 bp, Streptococcus pyogenes
ccaccccaaccccagttaa

SEQ ID NO: 2 - DNA, 20 bp, Streptococcus pyogenes
ggcggacatgcctttgttat

SEQ ID NO: 3 - DNA, 30 bp, Artificial Sequence
  Synthetic oligonucleotide
atggtagaagttacgtccgtcagcaccatc

SEQ ID NO: 4 - DNA, 19 bp, Streptococcus pyogenes
ggtggggttggggtcaatt

SEQ ID NO: 5 - DNA, 20 bp, Streptococcus pyogenes
ccgcctgtacggaaacaata

SEQ ID NO: 6 - DNA, 30 bp, Artificial Sequence
  Chemically Synthesized
taccatcttcaatgcaggcagtcgtggtag

(19) United States
(12) Patent Application Publication
(10) Pub. No.: US 2011/0045468 A1
(43) Pub. Date: Feb. 24, 2011

(54) Polynucleotides for the identification and quantification of group a streptococcus nucleic acids

(76) Inventors: Richard Robison, Provo, UT (US); Eldon G. Prince, Austin, TX (US); Benjamin Ari Satterfield, Galveston, TX (US)

Correspondence Address: Jacob C. Ong, 729 W Rolling Sage Way, Saratoga Springs, UT, 84045, US

(21) Appl. No.: 12/660,631

(22) Filed: Mar. 2, 2010

Related U.S. Application Data
(60) Provisional application No. 61/209,035, filed on Mar. 2, 2009.

(51) Int. Cl. C12Q 1/68; C07H 1/00; C12P 19/34

(52) U.S. Cl. 435/6; 536/24.32; 536/24.33; 435/91.1

(57) ABSTRACT

The present invention provides polynucleotides that can specifically hybridize to Group A Streptococcus (GAS) nucleic acids from all genotypes. These polynucleotides can be used in genotype-independent detection and quantitation of GAS nucleic acids. For example, the polynucleotides can be used as primers and/or probes in amplification-based assays for either end-point detection or real-time monitoring of GAS nucleic acids in a test sample. The polynucleotides can additionally be provided as part of a kit for the detection and quantitation of GAS nucleic acids.

### CROSS REFERENCE TO RELATED APPLICATION

This application claims the benefit under 35 U.S.C. §119(e) of U.S. Provisional Patent Application No. 61/209,305, filed Mar. 2, 2009, which is incorporated, in its entirety, by this reference.

### REFERENCE TO SEQUENCE LISTING

This specification incorporates by reference the material in the file that is named “upload_sequence.txt”, was created on May 11, 2010, is 2 kilobytes large, and was uploaded to the USPTO's EFS website on May 11, 2010. No new matter was added to the DNA sequence that was listed by paper and attached to the specification that was filed on Mar. 2, 2010. This application contains a Sequence Listing that follows the abstract.

### BACKGROUND OF THE INVENTION

### Field of the Invention

The present invention relates generally to the field of bacteria detection. The present invention provides polynucleotides that can specifically hybridize to Group A Streptococcus (GAS) nucleic acids from various genotypes. These polynucleotides can be used in genotype-independent detection and quantitation of GAS nucleic acids. For example, the polynucleotides can be used as primers and/or probes in amplification-based assays for either end-point detection or real-time monitoring of GAS nucleic acids in a test sample. The polynucleotides can additionally be provided as part of a kit for the detection and quantitation of GAS nucleic acids.

### BACKGROUND

A wide variety of biological research and clinical techniques utilize synthetic nucleic acid or other nucleobase polymer probes and primers for the detection, quantification, and characterization of infectious diseases from infectious agents, such as bacteria, fungi, virus or other organisms. Such techniques typically rely upon hybridization of the nucleic acid probes and primers to complementary regions of DNA or RNA that characterize the disease.

Streptococcus pyogenes (S. pyogenes), also known as Group A Streptococcus, is the etiologic agent of a number of infections in humans. S. pyogenes infections are of particular concern because serious complications may result if left untreated. Presumptive identification of S. pyogenes has been traditionally based upon physiological and biochemical traits; however, detection by classical techniques, such as culture and serotological methods can be indeterminate. Molecular assays are inherently valuable because detection can be achieved with enhanced sensitivity and specificity, and detection is not diminished with nonviable organisms.

### SUMMARY OF THE INVENTION

The invention discloses kits and methods for detecting at least one Group A Streptococcus bacterium comprising:
at least one forward primer, wherein said at least one forward primer is selected from the group consisting of oligonucleotides with the DNA sequence of SEQ ID NO: 1, an oligonucleotide sequence that is configured to hybridize with the DNA sequence of SEQ ID NO: 4, and an oligonucleotide sequence that is configured to be complementary with the DNA sequence of SEQ ID NO: 4; and,
at least one reverse primer, wherein said at least one reverse primer is selected from the group consisting of an oligonucleotide with the DNA sequence of SEQ ID NO: 2, at least one oligonucleotide sequence that is configured to hybridize with the DNA sequence of SEQ ID NO: 5, and at least one oligonucleotide sequence that is configured to be complementary with the DNA sequence of SEQ ID NO:4.
   The invention includes a kit, wherein said at least one forward primer is configured to hybridize with the DNA sequence of SEQ ID NO:4 under conditions suitable for polymerase chain reaction and said at least one reverse primer is configured to hybridize with the DNA sequence of SEQ ID NO:5 under conditions suitable for polymerase chain reaction.
   The invention also includes a kit wherein said at least one forward primer comprises oligonucleotides with the DNA sequence of SEQ ID NO:1 and said at least one reverse primer comprises oligonucleotides with the DNA sequence of SEQ ID NO:2.
   The invention also includes a kit further comprising at least one probe, wherein said at least one probe is configured to hybridize with at least one portion of a DNA sequence of a Group A Streptococcus bacterium selected from the group consisting of:
   1) a second DNA sequence, wherein said second DNA sequence is configured to be flanked on a first end by a third DNA sequence, said third DNA sequence being configured to be complementary with said at least one forward primer, wherein said second DNA sequence is further configured to be flanked on a second end by a fourth DNA sequence, said fourth DNA sequence being configured to correspond with said at least one reverse primer, and
   2) a fifth DNA sequence, wherein said fifth DNA sequence is configured to be flanked on a first end by a sixth DNA sequence, said sixth DNA sequence being configured to complement said at least one reverse primer, wherein said fifth DNA sequence is configured to be flanked on a second end by a seventh DNA sequence, said seventh DNA sequence being configured to correspond with said at least one forward primer.
      The invention includes a kit wherein said at least one probe comprises a nucleotide sequence, said nucleotide sequence comprising at least nine nucleotides.
      The invention includes a kit further comprising a probe, said probe comprising a DNA sequence of SEQ ID NO:3.
      The invention includes a kit further comprising a probe, wherein said probe comprises a DNA sequence of SEQ ID NO:3, wherein said at least one forward primer has the DNA sequence of SEQ ID NO: 1, and wherein said at least one reverse primer has the DNA sequence of SEQ ID NO:2.
      The invention includes a kit wherein said at least one forward primer and said at least one reverse primer are configured to amplify a portion of at least one DNA strand of at least one Group A Streptococcus bacterium.
      The invention includes a kit, further comprising at least one lysing solution, at least one buffer, and at least one solution comprising dNTPs.
      The invention also encompasses a method for forming at least one oligonucleotide, comprising:
      selecting at least one sequence from a group comprising: SEQ ID NO:1, SEQ ID NO:2, SEQ ID NO:3, SEQ ID NO:4, SEQ ID NO:5, SEQ ID NO:6, a seventh sequence,
      wherein said seventh sequence is configured to correspond with said SEQ ID NO: 1, an eighth sequence, wherein said eighth sequence is configured to correspond with said SEQ ID NO:2, a ninth sequence, wherein said ninth sequence is configured to correspond with said SEQ ID NO:3, a tenth sequence, wherein said tenth sequence is configured to correspond with said SEQ ID NO:4, an eleventh sequence, wherein said eleventh sequence is configured to correspond with said SEQ ID NO:5, a twelfth sequence, wherein said twelfth sequence is configured to correspond with said SEQ ID NO:6, and
      synthesizing said at least one sequence.
      A method for detecting the presence or absence of at least one Group A Streptococcus bacterium in a biological sample, comprising:

providing at least one sample, said at least one sample comprising at least one target DNA sequence of at least one Group A Streptococcus bacterium,

providing at least one DNA polymerase,

providing at least one forward primer selected from the group consisting of 1) the DNA sequence of SEQ ID NO: 1, 2) a second DNA sequence, wherein said second DNA sequence is configured to hybridize with the DNA sequence of SEQ ID NO:4 under conditions suitable for polymerase chain reaction, and 3) a third DNA sequence, wherein said third DNA sequence is configured to be complementary with the DNA sequence of SEQ ID NO:4,

providing at least one reverse primer selected from the group consisting of 1) the DNA sequence of SEQ ID NO: 2, 2) a fourth DNA sequence, wherein said fourth DNA sequence is configured to hybridize with the DNA sequence of SEQ ID NO:5 under conditions suitable for polymerase chain reaction, and 3) a fifth DNA sequence, wherein said fifth DNA sequence is configured to be complementary with the DNA sequence of SEQ ID NO:5,

providing at least one probe, wherein said at least one probe comprises at least one fluorophore, said at least one oligonucleotide, and said at least one quenching molecule, wherein said at least one fluorophore is configured to be linked with said at least one oligonucleotide of said probe and said quenching molecule is configured to be linked with said at least one oligonucleotide of said probe, wherein said at least one oligonucleotide is configured to hybridize with at least one portion of said target DNA sequence,
initiating a real-time PCR assay of a mixture comprising said at least one sample, said at least one DNA polymerase, said at least one forward primer, said at least one reverse primer, and said at least one probe, and,
amplifying said at least one DNA sequence of said at least one Group A Streptococcus bacterium, wherein at least one amplification product is formed,
contacting said at least one oligonucleotide with said DNA polymerase, wherein said DNA polymerase degrades said at least one oligonucleotide and disconnects said fluorophore from at least one object selected from the group consisting of said at least one oligonucleotide and said quenching molecule,
detecting at least one of the following scenarios selected from the group consisting of: 1) the presence of said amplification product, wherein the detection of said fluorophore signals the presence of said amplification product and said Group A Streptococcus bacterium, and 2) the absence of said amplification product, wherein the absence of said fluorophore signals the absence of said amplification product and said Group A Streptococcus bacterium.
   A method wherein said at least one fluorophore is selected from the group consisting of at least one fluorescein amidite, at least one fluorescein phosphoamidite, and at least one fluorescent molecule, wherein said quenching molecule is selected from the group consisting of at least one black hole quencher and at least one quenching molecule.
   A method wherein said at least one fluorophore is configured to be separated from said at least one quenching molecule by a calculated distance, wherein said calculated distance is sufficiently small so that said at least one quenching molecule quenches a fluorescent emission of said at least one fluorophore.
   A method further comprising the steps of designing a probe, synthesizing said probe, and implementing said probe in said real-time PCR, wherein said designing of said probe comprises the steps of:
   retrieving the DNA sequence of S. pyrogenes from a database,
   finding within said DNA sequence of S. pyrogenes a first target sequence and a second target sequence, wherein said first target sequence is flanked on a first end by a DNA sequence complementary to said at least one forward primer, wherein said DNA target sequence is flanked on a second end by a DNA sequence corresponding to said at least one reverse primer, wherein said second target sequence is complementary to said first target sequence,
   designing a probe comprising at least one oligonucleotide, at least one fluorophore, and at least one quenching molecule, wherein said at least one oligonucleotide is configured to hybridize with a sequence selected from the group consisting of: at least one portion of the first target sequence and at least one portion of the second target sequence.

### DETAILED DESCRIPTION OF THE INVENTION

Units, prefixes, and symbols may be denoted in their SI accepted form. Unless otherwise indicated, nucleic acids are written left to right in 5′ to 3′ orientation. Numeric ranges recited herein are inclusive of the numbers defining the range and include and are supportive of each integer within the defined range. Nucleotides may be referred to by their commonly accepted single-letter codes. Unless otherwise noted, the terms “a” or “an” are to be construed as meaning “at least one of”. The section headings used herein are for organizational purposes only and are not to be construed as limiting the subject matter described. All documents, or portions of documents, cited in this application, including but not limited to patents, patent applications, articles, books, and treatises, are herein expressly incorporated by reference in their entirety for any purpose.

The foregoing techniques and procedures are generally performed according to conventional methods well known in the arts of analytical chemistry, synthetic organic chemistry, and biochemistry and as described in various general and more specific references. See e.g., Sambrook et al. Molecular Cloning: A Laboratory Manual (2d ed., Cold Spring Harbor Laboratory Press, Cold Spring Harbor, N.Y. (1989)), which is incorporated herein by reference. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

### DEFINITIONS

“Complementary” means that a nucleobase of a polynucleotide is capable of hybridizing to a corresponding nucleobase in a different polynucleotide. As used herein, the term “complementary” is not limited to canonical Watson-Crick base pairs with A/T, G/C and U/A. Thus, nucleobase pairs may be considered to be “complementary” if one or both of the nucleobases is a nucleobase other than A, G, C, or T, such as a universal or degenerate nucleobase. A degenerate or universal nucleobase that is “complementary” to two or more corresponding nucleobases is considered to hybridize equivalently to the two or more corresponding nucleobases. The term “complementary” also refers to antiparallel strands of polynucleotides (as opposed to a single nucleobase pair) that are capable of hybridizing. For example, the sequence 5′-AGTTC-3′ is complementary to the sequence 5′-GAACT-3′. The term “complementary” is sometimes used interchangeably with “antisense.” Thus, degenerate nucleobase oligomers are said to hybridize to a corresponding multi-allelic polynucleotide template. The term “complementary.” as used in reference to two nucleotide sequences or two nucleobases, implies that the nucleotides sequences or nucleobases are “corresponding.”

“Corresponding” means, as between two nucleotide sequences or two nucleobases within a sequence, having the same or nearly the same relationship with respect to position and complementarity, or having the same or nearly the same relationship with respect to structure, function, or genetic coding (for example, as between a gene and the “corresponding” protein encoded by the gene). For example, a nucleotide sequence “corresponds” to a region of a polynucleotide template if the two sequences are complementary or have portions that are complementary. Similarly, a nucleobase of an oligomer “corresponds” to a nucleobase of a polynucleotide template when the two nucleobases occupy a position such that when the oligomer and the polynucleotide hybridize the two nucleobases pair opposite each other. The term “corresponding” is generally used herein in reference to the positional relationship between two polynucleotide sequences or two nucleobases. The term “corresponding” does not imply complementarity; thus, corresponding nucleobases may be complementary, or may be non-complementary.

“Nucleic acid” is a nucleobase polymer having a backbone formed from nucleotides, or nucleotide analogs. “Nucleic acid” and “polynucleotide” are considered to be equivalent and interchangeable, and refer to polymers of nucleic acid bases comprising any of a group of complex compounds composed of purines, pyrimidines, carbohydrates, and phosphoric acid. Nucleic acids are commonly in the form of DNA or RNA. The term “nucleic acid” includes polynucleotides of genomic DNA or RNA, cDNA, semisynthetic, or synthetic origin. Nucleic acids may also substitute standard nucleotide bases with nucleotide isoform analogs, including, but not limited to iso-C and iso-G bases, which may hybridize more or less permissibly than standard bases, and which will preferentially hybridize with complementary isoform analog bases. Many such isoform bases are described, for example, at www.idtdna.com. The nucleotides adenosine, cytosine, guanine and thymine are represented by their one-letter codes A, C, G, and T respectively. In representations of degenerate primers or mixture of different strands having mutations in one or several positions, the symbol R refers to either G or A, the symbol Y refers to either T/U or C, the symbol M refers to either A or C, the symbol K refers to either G or T/U, the symbol S refers to G or C, the symbol W refers to either A or T/U, the symbol B refers to “not A”, the symbol D refers to “not C”, the symbol H refers to “not G”, the symbol V refers to “not T/U” and the symbol N refers to any nucleotide.

“Nucleotide” refers to a phosphate ester of a nucleoside, as a monomer unit or within a polynucleotide polymer. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g., .alpha.-thio-nucleotide 5′-triphosphates. For a review of polynucleotide and nucleic acid chemistry, see Shabarova, Z. and Bogdanov, A. Advanced Organic Chemistry of Nucleic Acids, VCH, New York, 1994.

“Polymorphic site” means a base position of a polynucleotide “Polynucleotide” and “oligonucleotide” are used interchangeably and mean single-stranded and double-stranded polymers of nucleotide monomers, including 2′-deoxyribonucleotides (DNA) and ribonucleotides (RNA) linked by internucleotide phosphodiester bond linkages, e.g., 3′-5′ and 2′-5′, inverted linkages, e.g., 3′-3′ and 5′-5′, branched structures, or internucleotide analogs. A “polynucleotide sequence” refers to the sequence of nucleotide monomers along the polymer. “Polynucleotides” are not limited to any particular length of nucleotide sequence, as the term “polynucleotides” encompasses polymeric forms of nucleotides of any length. Polynucleotides that range in size from about 5 to about 40 monomeric units are typically referred to in the art as oligonucleotides. Polynucleotides that are several thousands or more monomeric nucleotide units in length are typically referred to as nucleic acids. Polynucleotides can be linear, branched linear, or circular molecules. Polynucleotides also have associated counter ions, such as H⁺, NH^{4′}, trialkylammonium, Mg²⁺, Na⁺ and the like.

Polynucleotides that are formed by 3′-5′ phosphodiester linkages are said to have 5′-ends and 3′-ends because the mononucleotides that are reacted to make the polynucleotide are joined in such a manner that the 5′ phosphate of one mononucleotide pentose ring is attached to the 3′ oxygen (i.e., hydroxyl) of its neighbor in one direction via the phosphodiester linkage. Thus, the 5′-end of a polynucleotide molecule has a free phosphate group or a hydroxyl at the 5′ position of the pentose ring of the nucleotide, while the 3′ end of the polynucleotide molecule has a free phosphate or hydroxyl group at the 3′ position of the pentose ring. Within a polynucleotide molecule, a position or sequence that is oriented 5′ relative to another position or sequence is said to be located “upstream,” while a position that is 3′ to another position is said to be “downstream.” This terminology reflects the fact that polymerases proceed and extend a polynucleotide chain in a 5′ to 3′ fashion along the template strand.

A polynucleotide may be composed entirely of deoxyribonucleotides, entirely of ribonucleotides, or chimeric mixtures thereof. Polynucleotides may be comprised of internucleotide, nucleobase and sugar analogs. Unless denoted otherwise, whenever a polynucleotide sequence is represented, it will be understood that the nucleotides are in 5′ to 3′ orientation from left to right and that “A” denotes deoxyadenosine, “C” denotes deoxycytidine, “G” denotes deoxyguanosine, and “T” denotes thymidine.

“Polynucleotide template” means the region of a polynucleotide complementary to an oligomer, probe or primer polynucleotide. It is understood that a polynucleotide template will normally constitute a portion of a larger polynucleotide molecule, with the “template” merely referring to that portion of the polynucleotide molecule to which the oligomer, probe or primer of the present invention is complementary. The term “template” thus refers to the region of the polynucleotide that constitutes the physical template for hybridization of another complementary polynucleotide. Templates may be genomic DNA, cDNA, PCR amplified DNA, or any other polynucleotide that serves as a pattern for the synthesis of a complementary polynucleotide.

“Primer” means an oligonucleotide molecule that is complementary to a portion of a target sequence and, upon hybridization to the target sequence, has a free 3′-hydroxyl group available for polymerase-catalyzed covalent bonding with a 5′-triphosphate group of a deoxyribonucleoside triphosphate molecule, thereby initiating the enzymatic polymerization of nucleotides complementary to the template. Primers may include detectable labels for use in detecting the presence of the primer or primer extension products that include the primer.

“Probe” refers to a nucleobase oligomer that is capable of forming a duplex structure by complementary base pairing with a sequence of a target polynucleotide, and further where the duplex so formed is detected, visualized, measured and/or quantitated. In some embodiments, the probe is fixed to a solid support, such as in column, a chip or other array format. Probes may include detectable labels for use in detecting the presence of the probe.

The practice of the present invention will employ, unless otherwise indicated, conventional techniques of molecular biology, microbiology, recombinant DNA techniques, and oligonucleotide synthesis which are within the skill of the art. Such techniques are explained fully in the literature. Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)); Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.), the contents of all of which are incorporated herein by reference.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a particular probe is disclosed and discussed and a number of modifications that can be made to a number of molecules including the probe are discussed, specifically contemplated is each and every combination and permutation of probes and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. For example CCACCCCAACCCCAGTTAA (SEQ ID NO: 1), GGCGGACATGCCTTTGTTAT (SEQ ID NO: 2) and 5′-FAM-ATGGTAGAAGTTACGTCCGTCAGCACCATC-3BHQ1-3′ (SEQ ID NO: 3) set forth particular sequences of a primer set and a probe, respectively, for specific and sensitive amplification and detection of a target area on GSA. Included herein within the scope of the invention are variants of these and other genes and proteins herein disclosed which have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

2. Hybridization/Selective Hybridization

The term hybridization typically means a sequence driven interaction between at least two nucleic acid molecules, such as a primer or a probe and a gene or a portion of a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k.sub.d, or where only one of the nucleic acid molecules is 10 fold or 100 fold or 1000 fold or where one or both nucleic acid molecules are above their k.sub.d.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically-manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein. Examples of specific hybridization conditions are provided herein. For the reasons stated above, these conditions are exemplary only and do not limit the real-time PCR method described.

3. Nucleic Acids

There are a variety of molecules disclosed herein that are nucleic acid based, including for example the primers and probe that hybridize specifically to the target area of GAS. The disclosed nucleic acids are made up of for example, nucleotides, nucleotide analogs, or nucleotide substitutes. Non-limiting examples of these and other molecules are discussed herein. It is understood that for example, when a vector is expressed in a cell that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (psi.), hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C.sub.1 to C.sub.10, alkyl or C.sub.2 to C.sub.10 alkenyl and alkynyl. 2′ sugar modifications also include but are not limited to —O[(CH.sub.2).sub.nO].sub.mCH.sub.3, —O(CH.sub.2).sub.nOCH.sub.3, —O(CH.sub.2).sub.nNH.sub.2, —O(CH.sub.2).sub.nCH.sub.3, —O(CH.sub.2).sub.n-ONH.sub.2, and —O(CH.sub.2).sub.nON[(CH.sub.2).sub.nCH.sub.3)].sub.2, where n and m are from 1 to about 10.

Other modifications at the 2′ position include but are not limited to: C.sub.1 to C.sub.10 lower alkyl, substituted lower alkyl, alkaryl, aralkyl, O-alkaryl or O-aralkyl, SH, SCH.sub.3, OCN, Cl, Br, CN, CF.sub.3, OCF.sub.3, SOCH.sub.3, SO.sub.2 CH.sub.3, ONO.sub.2, NO.sub.2, N.sub.3, NH.sub.2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH.sub.2 and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science, 1991, 254, 1497-1500).

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

One particular sequence set forth in SEQ. ID. NO. 1 is used herein, as an example of a disclosed primer. One particular sequence set forth in SEQ ID NO: 2 is an example of an additional disclosed primer. One particular sequence set forth in SEQ ID NO: 3 is an example of a disclosed probe. Primers and/or probes can be designed to be specific for specific sequences given the information disclosed herein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

c) Primers and Probes

Disclosed are compositions including primers and probes, which are capable of interacting with the target area disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner.

Dark quenchers, black hole quenchers, and other quenchers are known in the art. Also disclosed in the prior art is a nondegenerate probe that is an oligonucleotide, comprising: 5′-X-CTAGCACATGC″T″ACAAGAATGATTGCAGAAAGAAA-Y-3′, wherein X is a fluorophore, wherein Y is a phosphate group or phosphate groups, wherein “T” is a thymine with a dark quencher or acceptor dye linked to it.

In some embodiments, the fluorophore can be carboxyfluorescein (HEX), Fam, Joe, 6-carboxy-X-rhodamine (Rox), Texas Red, or Cy 5.

Also, in some embodiments, 1, 2, 3, 4, 5, 6, or 7 phosphate groups can be attached to the 3′ end of the probe.

In some embodiments, the dark quencher is attached to the “T” residue of the probe can be a Black hole quencher (BHQ1-dT), Dabcyl-dT (Glen Research) or QSY7 (Molecular probes) via an aminolink modified-dT.

Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit comprising reagents for real-time PCR-type amplification reaction for detecting GAS, comprising sense primers, antisense primers and a nondegenerate probe. For example the kit can detect a target area of GAS.

The disclosed kits can include any of the probes as defined herein, for example a probe having a fluorophore attached to the 5′ end of the probe, wherein at least one phosphate group is attached to the 3′ end of the probe, and wherein a dark quencher is attached to the “T” residue of the probe, falls within the scope of the invention.

Also disclosed is a kit comprising reagents for PCR-type amplification reaction for detecting GAS, comprising sense primers, antisense primers and a nondegenerate probe wherein the sense primer is an oligonucleotide comprising SEQ ID NO: 1 or a sequence that hybridizes, under conditions suitable for a polymerase chain reaction, with: SEQ ID NO: 5; or a sequence complementary thereto, wherein the oligonucleotide is from 9-40 consecutive nucleotides.

Also disclosed is a kit comprising reagents for real-time PCR-type amplification reaction for detecting GAS, comprising sense primers, antisense primers and a nondegenerate probe wherein the antisense primer is an oligonucleotide, comprising at least 9 consecutive nucleotides of SEQ ID NO: 2 or a sequence that hybridizes, under conditions suitable for a polymerase chain reaction, with: SEQ ID NO: 6; or a sequence complementary thereto.

Also disclosed is a kit comprising reagents for real-time PCR-type amplification reaction for detecting GAS, comprising sense primers, antisense primers and a probe wherein the probe is an oligonucleotide, comprising at least 20 consecutive nucleotides of SEQ ID NO: 3 or a sequence that hybridizes, under conditions suitable for a polymerase chain reaction, with: SEQ ID NO: 6; or a sequence complementary thereto. The disclosed kits can include any of the probes as defined herein, for example a probe having a fluorophore attached to the 5′ end of the probe, wherein at least one phosphate group is attached to the 3′ end of the probe, and wherein a dark quencher is attached to the “T” residue of the probe.

8. Compositions with Similar Functions

### C. METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1 Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Process for Making the Compositions

Disclosed are processes for making the compositions as well as making the intermediates leading to the compositions. For example, disclosed are nucleic acids in SEQ ID NOS: 1, 2, and 3.

There are a variety of methods that can be used for making these compositions, such as synthetic chemical methods and standard molecular biology methods. It is understood that the methods of making these and the other disclosed compositions are specifically disclosed.

Also disclosed are nucleic acid molecules produced by the process comprising linking in an operative way a nucleic acid molecule comprising a sequence having at least 80% identity to a sequence set forth in SEQ ID NOS: 1, 2, and 3, and a sequence controlling the expression of the nucleic acid.

Also disclosed are nucleic acid molecules produced by the process comprising linking in an operative way a nucleic acid molecule comprising SEQ ID NO: 3 to a fluorophore on the 5′ end. It is known in the art that a fluorophore and a quencher can be embedded in a nucleotide sequence and does not necessarily have to be linked to the ends of the sequence.

A dark quencher molecule can be linked to the probe by an amino linkage, however any standard method of attaching a dark quencher to an internal “T” residue can be used in this method.

### D. METHODS OF USING THE COMPOSITIONS

1. Methods of Using the Compositions as Research Tools

The disclosed compositions, either alone or in combination, can be used in a variety of ways. For example, the disclosed compositions, such as SEQ ID NOS: 1, 2, and 3 either alone or in combination can be used to detect the presence of the target area.

The compositions, either alone or in combination, can also be used to detect GAS serotypes, for example, GAS infections.

The disclosed compositions, either alone or in combination, can also be used to differentiate between true Streptococcus pyrogenes species and similar species.

The disclosed compositions, either alone or in combination, can also be used as compositions for carrying out a polymerase chain reaction (PCR).

The disclosed compositions, either alone or in combination, can also be used as compositions for carrying out a real-time PCR reaction.

The disclosed compositions, either alone or in combination, can also be used to differentially detect the presence true GAS from similar species.

a) Polymerase Chain Reaction (PCR)

The technology of PCR permits amplification and subsequent detection of minute quantities of a target nucleic acid. Details of PCR are well described in the art, including, for example, U.S. Pat. Nos. 4,683,195 to Mullis et al., U.S. Pat. No. 4,683,202 to Mullis and U.S. Pat. No. 4,965,188 to Mullis et al. Generally, oligonucleotide primers are annealed to the denatured strands of a target nucleic acid, and primer extension products are formed by the polymerization of deoxynucleoside triphosphates by a polymerase. A typical PCR method involves repetitive cycles of template nucleic acid denaturation, primer annealing and extension of the annealed primers by the action of a thermostable polymerase. The process results in exponential amplification of the target nucleic acid, and thus allows the detection of targets existing in very low concentrations in a sample. PCR is widely used in a variety of applications, including biotechnological research, clinical diagnostics and forensics.

b) Real-Time PCR

In implementing the present invention, reference may optionally be made to a general review of PCR techniques and to the explanatory note entitled “Quantitation of DNA/RNA Using Real-Time PCR Detection” published by Perkin Elmer Applied Biosystems (1999) and to PCR Protocols (Academic Press New York, 1989).

Real-time PCR monitors the fluorescence emitted during the reaction as an indicator of amplicon production during each PCR cycle (ie, in real time) as opposed to the endpoint detection (For example see FIG. 1 of U.S. Pat. No. 7,476,733; Higuchi, 1992; Higuchi, 1993). The real-time progress of the reaction can be viewed in some systems.

The real-time PCR system is based on the detection of a fluorescent reporter (Lee, 1993; Livak, 1995). This signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. The higher the starting copy number of the nucleic acid target, the sooner a significant increase in fluorescence is observed.

There are three main fluorescence-monitoring systems for DNA amplification (Wittwer, 1997(a)): (1) hydrolysis probes; (2) hybridising probes (see Hybridisation Probe Chemistry, incorporated herein by reference for its teaching of fluorescence monitoring systems); and (3) DNA-binding agents (Wittwer, 1997; van der Velden, 2003, incorporated herein for their teaching of DNA-binding agents). Hydrolysis probes include TaqMan™ probes (Heid et al, 1996, incorporated herein by reference for its teaching of hydrolysis probes), molecular beacons (Mhlanga, 2001; Vet, 2002; Abravaya, 2003; Tan, 2004; Vet & Marras, 2005, incorporated herein by reference for their teaching of molecular beacons) and scorpions (Saha, 2001; Solinas, 2001; Terry, 2002, incorporated herein by reference for their teaching of scorpions). They use the fluorogenic 5′ exonuclease activity of Taq polymerase to measure the amount of target sequences in cDNA samples (see also Svanvik, 2000, incorporated herein by reference for its teaching of light-up probes).

TaqMan® probes are oligonucleotides longer than the primers (20-30 bases long with a Tm value of 10° C. higher) that contain a fluorescent dye usually on the 5′ base, and a quenching dye typically on the 3′ base. When irradiated, the excited fluorescent dye transfers energy to the nearby quenching dye molecule (this is called FRET=Forster or fluorescence resonance energy transfer) (Hiyoshi, 1994; Chen, 1997). Thus, the close proximity of the reporter and quencher prevents detection of any fluorescence while the probe is intact. TaqMan® probes are designed to anneal to an internal region of a PCR product. When the polymerase replicates a template on which a TaqMan® probe is bound, its 5′ exonuclease activity cleaves the probe (Holland, 1991). This ends the activity of quencher (no FRET) and the reporter dye starts to emit fluorescence which increases in each cycle proportional to the rate of probe cleavage. Accumulation of PCR products is detected by monitoring the increase in fluorescence of the reporter dye (note that primers are not labelled). TaqMan® assay uses universal thermal cycling parameters and PCR reaction conditions. Because the cleavage occurs only if the probe hybridises to the target, the origin of the detected fluorescence is specific amplification. The process of hybridisation and cleavage does not interfere with the exponential accumulation of the product. One specific requirement for fluorogenic probes is that there be no G at the 5′ end. A ‘G’ adjacent to the reporter dye can quench reporter fluorescence even after cleavage.

Molecular beacons also contain fluorescent (FAM, TAMRA, TET, ROX) and quenching dyes (typically DABCYL) at either end but they are designed to adopt a hairpin structure while free in solution to bring the fluorescent dye and the quencher in close proximity for FRET to occur. They have two arms with complementary sequences that form a very stable hybrid or stem. The close proximity of the reporter and the quencher in this hairpin configuration suppresses reporter fluorescence. When the beacon hybridises to the target during the annealing step, the reporter dye is separated from the quencher and the reporter fluoresces (FRET does not occur). Molecular beacons remain intact during PCR and must rebind to target every cycle for fluorescence emission. This will correlate to the amount of PCR product available. All real-time PCR chemistries allow detection of multiple DNA species (multiplexing) by designing each probe/beacon with a spectrally unique fluor/quench pair as long as the platform is suitable for melting curve analysis. By multiplexing, the target(s) and endogenous control can be amplified in single tube. For examples, see Bernard, 1998; Vet, 1999; Lee, 1999; Donohoe, 2000; Read, 2001; Grace, 2003; Vrettou, 2004; Rickert, 2004.

With Scorpion probes, sequence-specific priming and PCR product detection is achieved using a single oligonucleotide. The Scorpion probe maintains a stem-loop configuration in the unhybridised state. The fluorophore is attached to the 5′ end and is quenched by a moiety coupled to the 3′ end. The 3′ portion of the stem also contains sequence that is complementary to the extension product of the primer. This sequence is linked to the 5′ end of a specific primer via a non-amplifiable monomer. After extension of the Scorpion primer, the specific probe sequence is able to bind to its complement within the extended amplicon thus opening up the hairpin loop. This prevents the fluorescence from being quenched and a signal is observed.

Another alternative is the double-stranded DNA binding dye chemistry, which quantitates the amplicon production (including non-specific amplification and primer-dimer complex) by the use of a non-sequence specific fluorescent intercalating agent (SYBR-green I or ethidium bromide). It does not bind to ssDNA. SYBR green is a fluorogenic minor groove binding dye that exhibits little fluorescence when in solution but emits a strong fluorescent signal upon binding to double-stranded DNA (Morrison, 1998). Disadvantages of SYBR green-based real-time PCR include the requirement for extensive optimisation. Furthermore, non-specific amplifications require follow-up assays (melting point curve or dissociation analysis) for amplicon identification (Ririe, 1997). The method has been used in HFE-C282Y genotyping (Donohoe, 2000). Another controllable problem is that longer amplicons create a stronger signal (if combined with other factors, this may cause CCD camera saturation, see below). Normally SYBR green is used in singleplex reactions, however when coupled with melting point analysis, it can be used for multiplex reactions (Siraj, 2002).

The threshold cycle or the CT value is the cycle at which a significant increase in ARn is first detected (for definition of ARn, see below). The threshold cycle is when the system begins to detect the increase in the signal associated with an exponential growth of PCR product during the log-linear phase. This phase provides the most useful information about the reaction (certainly more important than the end-point). The slope of the log-linear phase is a reflection of the amplification efficiency. The efficiency (Eff) of the reaction can be calculated by the formula: Eff=10.sup. (−1/slope)-1. The efficiency of the PCR should be 90-110% (−3.6>slope>−3.1). A number of variables can affect the efficiency of the PCR. These factors include length of the amplicon, secondary structure and primer quality. Although valid data can be obtained that fall outside of the efficiency range, the real time PCR should be further optimised or alternative amplicons designed. For the slope to be an indicator of real amplification (rather than signal drift), there has to be an inflection point. This is the point on the growth curve when the log-linear phase begins. It also represents the greatest rate of change along the growth curve. (Signal drift is characterised by gradual increase or decrease in fluorescence without amplification of the product.) The important parameter for quantitation is the C.sub.T. The higher the initial amount of genomic DNA, the sooner accumulated product is detected in the PCR process, and the lower the C.sub.T value. The threshold should be placed above any baseline activity and within the exponential increase phase (which looks linear in the log transformation). Some software allows determination of the cycle threshold (C.sub.T) by a mathematical analysis of the growth curve. This provides better run-to-run reproducibility. Besides being used for quantitation, the C.sub.T value can be used for qualitative analysis as a pass/fail measure.

In some aspects of the real time PCR method disclosed, multiplex TaqMan® assays can be performed with ABI instruments using multiple dyes with distinct emission wavelengths. Available dyes for this purpose are FAM, TET, VIC and JOE (the most expensive). TAMRA is reserved as the quencher on the probe and ROX as the passive reference. For best results, the combination of FAM (target) and VIC (endogenous control) is recommended (they have the largest difference in emission maximum) whereas JOE and VIC should not be combined. It is important that if the dye layer has not been chosen correctly, the machine will still read the other dye's spectrum. For example, both VIC and FAM emit fluorescence in a similar range to each other and when doing a single dye, the wells should be labelled correctly. In the case of multiplexing, the spectral compensation for the post run analysis should be turned on (on ABI 7700: Instrument/Diagnostics/Advanced Options/Miscellaneous). Activating spectral compensation improves dye spectral resolution.

In addition, the real-time PCR reaction can be carried out in a wide variety of platforms including, but not limited to ABI 7700 (ABI), the LightCycler (Roche Diagnostics), iCycler (RioRad), DNA Engine Opticon ContinuousFluorescence Detection System (MI Research), Mx400 (Stratagene), Chimaera Quantitative Detection System (Thermo Hybaid), Rotor-Gene 3000 (Corbett Research), Smartcycler (Cepheid), or the MX3000P format (Stratagene).

Disclosed is a method for detecting GAS nucleic acid in a biological sample, comprising: producing an amplification product by amplifying an GAS nucleotide sequence using sense primers and antisense primers, wherein said primers are chosen from oligonucleotides that hybridize, under conditions suitable for a polymerase chain reaction, with a sequence of the target area of GAS; and detecting said amplification product, whereby the presence of GAS nucleic acid is detected.

Also disclosed is a method for detecting GAS nucleic acid in a biological sample, comprising: producing an amplification product by amplifying an GAS nucleotide sequence by real-time PCR using: a primer consisting of SEQ ID NO: 1 or a sequence that hybridizes, under conditions suitable for a polymerase chain reaction, with: SEQ ID NO: 5; or a sequence complementary thereto, and a primer consisting of SEQ ID NO: 2 or a sequence that hybridizes, under conditions suitable for a polymerase chain reaction, with: SEQ ID NO: 6; or a sequence complementary thereto, under conditions suitable for a polymerase chain reaction; and detecting said amplification product by using: a probe consisting of SEQ ID NO: 3 or a sequence that hybridizes, under conditions suitable for a polymerase chain reaction, with: SEQ ID NO: 7; or a sequence complementary thereto, that hybridizes, under conditions suitable for a polymerase chain reaction, whereby the presence of GAS nucleic acid is detected.

c) Quantifying GAS Nucleic Acid in a Biological Sample

The disclosed compositions, either alone or in combination, can also be used a method for quantifying GAS nucleic acid in a biological sample, comprising: producing an amplification product by amplifying an GAS nucleotide sequence by real-time PCR using sense primers and antisense primers, wherein said primers are chosen from oligonucleotides that hybridize, under conditions suitable for a polymerase chain reaction, with a sequence of the target area of GAS; and detecting said amplification product by using a nondegenerate probe comprising an oligonucleotide that hybridizes, under conditions suitable for a polymerase chain reaction, with a sequence of the target area of GAS; and quantifying said amplification product in said biological sample by measuring a detection signal from said probe and comparing said detection signal to a second probe detection signal from a quantification standard, wherein said quantification standard comprises a sense probe and a nucleic acid standard.

For all of the methods described herein, a biological sample can be from any organism and can be, but is not limited to serum, peripheral blood, bone marrow specimens, embedded tissue sections, frozen tissue sections, cell preparations, cytological preparations, exfoliate samples (e.g., sputum), fine needle aspirations, amnion cells, fresh tissue, dry tissue, and cultured cells or tissue. Such samples can be obtained directly from a subject, commercially obtained or obtained via other means. Thus, the invention described herein can be utilized to analyze a nucleic acid sample that comprises genomic DNA, amplified DNA (such as a PCR product) cDNA, cRNA, a restriction fragment or any other desired nucleic acid sample. When one performs one of the herein described methods on genomic DNA, typically the genomic DNA will be treated in a manner to reduce viscosity of the DNA and allow better contact of a primer or probe with the target region of the genomic DNA. Such reduction in viscosity can be achieved by any desired methods, which are known to the skilled artisan, such as DNase treatment or shearing of the genomic DNA, preferably lightly.

2. Methods of Using the Compositions as Diagnostic Tools

The disclosed compositions, either alone or in combination, can also be used diagnostic tools related to diseases, such as pneumococcal disease. For example, the disclosed compositions, such as SEQ ID NOS: 1, 2, and 3 can be used to diagnose GAS, by detecting the presence of the target area.

The disclosed compositions, either alone or in combination, can also be used in a method for detecting GAS nucleic acid in a biological sample, comprising: producing an amplification product by amplifying an GAS nucleotide sequence using sense primers and antisense primers, wherein said primers are chosen from oligonucleotides that hybridize, under conditions suitable for a polymerase chain reaction, with a sequence of the target area of GAS; and detecting said amplification product, whereby the presence of GAS nucleic acid is detected, wherein the detection of GAS nucleic acid diagnoses GAS infection.

The disclosed compositions, either alone or in combination, can also be used in a method for detecting GAS nucleic acid in a biological sample, comprising: producing an amplification product by amplifying an GAS nucleotide sequence by real-time PCR using: a primer consisting of SEQ ID NO: 1 or a sequence that hybridizes, under conditions suitable for a polymerase chain reaction, with: SEQ ID NO: 4; or a sequence complementary thereto, and a primer consisting of SEQ ID NO: 2 or a sequence that hybridizes, under conditions suitable for a polymerase chain reaction, with: SEQ ID NO: 5; or a sequence complementary thereto, under conditions suitable for a polymerase chain reaction; and detecting said amplification product by using: a probe consisting of SEQ ID NO: 3 or a sequence that hybridizes, under conditions suitable for a polymerase chain reaction, with: SEQ ID NO:6; or a sequence complementary thereto, wherein a fluorophore is attached to the 5′ end of the probe, wherein at least one phosphate group is attached to the 3′ end of the probe, and wherein a dark quencher is attached to the “T” residue of the probe, under conditions suitable for a polymerase chain reaction, whereby the presence of GAS nucleic acid is detected, wherein the detection of GAS nucleic acid diagnoses GAS infection.

The disclosed compositions can also be used in a method for detecting GAS nucleic acid in a biological sample, comprising: producing an amplification product by amplifying an GAS nucleotide sequence using a sense primer and a antisense primer, wherein said primers are chosen from oligonucleotides that hybridize, under conditions suitable for a polymerase chain reaction, with a sequence of the target area of GAS; and detecting said amplification product, whereby the presence of GAS nucleic acid is detected, wherein the sense primer consists of SEQ ID NO: 1 or a sequence that hybridizes, under conditions suitable for a polymerase chain reaction, with: SEQ ID NO: 5; or a sequence complementary thereto, wherein the detection of GAS nucleic acid diagnoses GAS infection.

The disclosed compositions can also be used in a method for detecting GAS nucleic acid in a biological sample, comprising: producing an amplification product by amplifying an GAS nucleotide sequence by real-time PCR using sense primers and antisense primers, wherein said primers are chosen from oligonucleotides that hybridize, under conditions suitable for a polymerase chain reaction, with a sequence of the target area of GAS; and detecting said amplification product, whereby the presence of GAS nucleic acid is detected, wherein the antisense primer consists of SEQ ID NO: 2 or a sequence that hybridizes, under conditions suitable for a polymerase chain reaction, with: SEQ ID NO: 5; or a sequence complementary thereto, wherein the detection of GAS nucleic acid diagnoses GAS infection.

The disclosed compositions can also be used in a method for detecting GAS nucleic acid in a biological sample using Real-Time PCR, comprising: producing an amplification product by amplifying an GAS nucleotide sequence using sense primers and antisense primers, wherein said primers are chosen from oligonucleotides that hybridize, under conditions suitable for a polymerase chain reaction, with a sequence of the target area of GAS; and detecting said amplification product, whereby the presence of GAS nucleic acid is detected, wherein the probe consists of SEQ ID NO: 3 or a sequence that hybridizes, under conditions suitable for a polymerase chain reaction, with: SEQ ID NO: 6; or a sequence complementary thereto, wherein a fluorophore is attached to the 5′ end of the probe, wherein at least one phosphate group is attached to the 3′ end of the probe, and wherein a dark quencher is attached to the “T” residue of the probe, wherein the detection of GAS nucleic acid diagnoses GAS infection. However, there are many different methods whereby a quenching molecule could be attached to the probe.

The disclosed compositions can also be used in a method for detecting GAS nucleic acid in a biological sample, comprising: producing an amplification product by amplifying an GAS nucleotide sequence by real-time PCR.

The disclosed compositions, either alone or in combination, can also be used to diagnose GAS, by detecting the presence of the target area in true GAS species. True GAS species are described elsewhere herein.

The disclosed compositions, either alone or in combination, can also be used to diagnose GAS, by detecting the presence of the target area in true GAS species in different serotypes.

The disclosed compositions, either alone or in combination, can also be used to differentially diagnose true GAS infection from GAS-like species infections.

3. Methods of Evaluating Expression of the Gene Using Micro Arrays

The disclosed compositions, either alone or in combination, can be used as discussed herein as either reagents in micro arrays or as reagents to probe or analyze existing microarrays.

4. Methods of Screening Assay Using a Chip/Micro Array

The disclosed compositions, either alone or in combination, can be used as discussed herein as either reagents in chips and micro arrays or as reagents to probe or analyze existing chips and microarrays.

5. Latex Agglutination

Latex agglutination is a well-established immunoassay method in which latex particles are coated with an analyte-specific capture reagent, such as an antibody. The major limitations of agglutination-based assays are their lack of sensitivity and specificity and the subjective nature of test result interpretation. However, because these tests are fast, inexpensive and require minimal reagents, they have been widely used.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in degree C. or is at ambient temperature, and pressure is at or near atmospheric.

### 1. Example 1

Group A Streptococcus (GAS) primers/probe

Forward primer:
CCACCCCAACCCCAGTTAA
Reverse primer:
GGCGGACATGCCTTTGTTA
Probe:
5′-FAM-ATGGTAGAAGTTACGTCCGTCAGCACCATC-3BHQ1-3′

Each primer and the probe were diluted in PCR H2O to 500 nm.

Protocol

Collection

Samples were collected using two swabs simultaneously on individual patients. One swab was used to perform a rapid latex bead test. The other swab was placed in a 15 ml tube with a 1 ml solution of Tris/EDTA to prevent degradation. Samples were then stored at 4 degrees C.

Stored swabs were streaked on tryptic soy agar (TSA) with blood agar plates to verify by culturing. Swab solutions were vortexed for about 10 seconds. 50 μl of the samples were placed into microcentrifuging tubes which were then boiled for approximately 2 minutes, though we found that this step can be eliminated. 21.25 μl of each sample was placed in tubes containing one GE Illustra™ Hot Start Mix RTG bead. 1.25 μl of each of the primers and the probe was added and then each sample was vortexed. These solutions were placed into 25 μl Cepheid tubes and PCR was performed on a Cepheid SmartCycler II. We optimized the PCR cycling conditions as follows:

Initial melting step of 95 degrees C. for 2 min 30 sec followed by 40 cycles of 61 degrees C. for 5 sec and 95 degrees C. for 10 sec.

Results

79 S. pyogenes isolates were obtained from the Intermountain Medical Center and the Timpanogos Regional Hospital. All of the isolates were confirmed using bacitracin sensitivity assays and gram staining. All 79 isolates tested positive with real time PCR using the above methods. ATCC strains 51339, 14289, and 49399 also were tested. To control these results we tested near neighbors B. thuringiensis, B. Subtilis, E. faecalis, P. polymxa, Faecalis, P. polymxa, C. botulinum, Bt. Kurstaki, S. pneumiae, E. coli, S. typhimirium, S. dysenteriae, S. cholerasius, P. aeruginosa, S. aureus, L. acidophilus, B. circulans, B. mycoides, B. anthracis, B. licheniformis, and 7 strains of S. agalactiae. All of these strains showed negative results using RT PCR.
200 patients were swabbed and tested by the Student Health Center using rapid latex bead tests. We tested these same patients using real time PCR. Of the 200 tested, 22 were observed to be positive by the Student Health Center. All of these strains tested positive using RT PCR. One of the 200 strains tested negative at the Student Health Center which tested positive using RT PCR. (Mother was infected and asked to have her baby tested.)

1. A kit for detecting at least one Group A Streptococcus bacterium comprising:
at least one forward primer, wherein said at least one forward primer is selected from the group consisting of oligonucleotides with the DNA sequence of SEQ ID NO: 1, an oligonucleotide sequence that is configured to hybridize with the DNA sequence of SEQ ID NO: 4, and an oligonucleotide sequence that is configured to be complementary with the DNA sequence of SEQ ID NO: 4; and,
at least one reverse primer, wherein said at least one reverse primer is selected from the group consisting of an oligonucleotide with the DNA sequence of SEQ ID NO: 2, at least one oligonucleotide sequence that is configured to hybridize with the DNA sequence of SEQ ID NO: 5, and at least one oligonucleotide sequence that is configured to be complementary with the DNA sequence of SEQ ID NO:4.

2. The kit of claim 1, wherein said at least one forward primer is configured to hybridize with the DNA sequence of SEQ ID NO:4 under conditions suitable for polymerase chain reaction and said at least one reverse primer is configured to hybridize with the DNA sequence of SEQ ID NO:5 under conditions suitable for polymerase chain reaction.

3. The kit of claim 1, wherein said at least one forward primer comprises oligonucleotides with the DNA sequence of SEQ ID NO:1 and said at least one reverse primer comprises oligonucleotides with the DNA sequence of SEQ ID NO:2.

4. The kit of claim 1, further comprising at least one probe, wherein said at least one probe is configured to hybridize with at least one portion of a DNA sequence of a Group A Streptococcus bacterium selected from the group consisting of:
1) a second DNA sequence, wherein said second DNA sequence is configured to be flanked on a first end by a third DNA sequence, said third DNA sequence being configured to be complementary with said at least one forward primer, wherein said second DNA sequence is further configured to be flanked on a second end by a fourth DNA sequence, said fourth DNA sequence being configured to correspond with said at least one reverse primer, and
2) a fifth DNA sequence, wherein said fifth DNA sequence is configured to be flanked on a first end by a sixth DNA sequence, said sixth DNA sequence being configured to complement said at least one reverse primer, wherein said fifth DNA sequence is configured to be flanked on a second end by a seventh DNA sequence, said seventh DNA sequence being configured to correspond with said at least one forward primer.

5. The kit of claim 3, wherein said at least one probe comprises a nucleotide sequence, said nucleotide sequence comprising at least nine nucleotides.

6. The kit of claim 3, further comprising a probe, said probe comprising a DNA sequence of SEQ ID NO:3.

7. The kit of claim 1, further comprising a probe, wherein said probe comprises a DNA sequence of SEQ ID NO:3, wherein said at least one forward primer has the DNA sequence of SEQ ID NO: 1, and wherein said at least one reverse primer has the DNA sequence of SEQ ID NO:2.

8. The kit of claim 1, wherein said at least one forward primer and said at least one reverse primer are configured to amplify a portion of at least one DNA strand of at least one Group A Streptococcus bacterium.

9. The kit of claim 4, further comprising at least one lysing solution, at least one buffer, and at least one solution comprising dNTPs.

10. A method for forming at least one oligonucleotide, comprising:
selecting at least one sequence from a group comprising: SEQ ID NO: 1, SEQ ID NO:2, SEQ ID NO:3, SEQ ID NO:4, SEQ ID NO:5, SEQ ID NO:6, a seventh sequence, wherein said seventh sequence is configured to correspond with said SEQ ID NO: 1, an eighth sequence, wherein said eighth sequence is configured to correspond with said SEQ ID NO:2, a ninth sequence, wherein said ninth sequence is configured to correspond with said SEQ ID NO:3, a tenth sequence, wherein said tenth sequence is configured to correspond with said SEQ ID NO:4, an eleventh sequence, wherein said eleventh sequence is configured to correspond with said SEQ ID NO:5, a twelfth sequence, wherein said twelfth sequence is configured to correspond with said SEQ ID NO:6,
synthesizing said at least one sequence.

11. A method for detecting the presence or absence of at least one Group A Streptococcus bacterium in a biological sample, comprising:
providing at least one sample, said at least one sample comprising at least one target DNA sequence of at least one Group A Streptococcus bacterium,
providing at least one DNA polymerase,
providing at least one forward primer selected from the group consisting of 1) the DNA sequence of SEQ ID NO: 1, 2) a second DNA sequence, wherein said second DNA sequence is configured to hybridize with the DNA sequence of SEQ ID NO:4 under conditions suitable for polymerase chain reaction, and 3) a third DNA sequence, wherein said third DNA sequence is configured to be complementary with the DNA sequence of SEQ ID NO:4,
providing at least one reverse primer selected from the group consisting of 1) the DNA sequence of SEQ ID NO: 2, 2) a fourth DNA sequence, wherein said fourth DNA sequence is configured to hybridize with the DNA sequence of SEQ ID NO:5 under conditions suitable for polymerase chain reaction, and 3) a fifth DNA sequence, wherein said fifth DNA sequence is configured to be complementary with the DNA sequence of SEQ ID NO:5,
providing at least one probe, wherein said at least one probe comprises at least one fluorophore, said at least one oligonucleotide, and said at least one quenching molecule, wherein said at least one fluorophore is configured to be linked with said at least one oligonucleotide of said probe and said quenching molecule is configured to be linked with said at least one oligonucleotide of said probe, wherein said at least one oligonucleotide is configured to hybridize with at least one portion of said target DNA sequence,
initiating a real-time PCR assay of a mixture comprising said at least one sample, said at least one DNA polymerase, said at least one forward primer, said at least one reverse primer, and said at least one probe, and,
amplifying said at least one DNA sequence of said at least one Group A Streptococcus bacterium, wherein at least one amplification product is formed,
contacting said at least one oligonucleotide with said DNA polymerase, wherein said DNA polymerase degrades said at least one oligonucleotide and disconnects said fluorophore from at least one object selected from the group consisting of said at least one oligonucleotide and said quenching molecule,
detecting at least one of the following scenarios selected from the group consisting of: 1) the presence of said amplification product, wherein the detection of said fluorophore signals the presence of said amplification product and said Group A Streptococcus bacterium, and 2) the absence of said amplification product, wherein the absence of said fluorophore signals the absence of said amplification product and said Group A Streptococcus bacterium.

13. The method of claim 12, wherein said at least one fluorophore is selected from the group consisting of at least one fluorescein amidite, at least one fluorescein phosphoamidite, and at least one fluorescent molecule, wherein said quenching molecule is selected from the group consisting of at least one black hole quencher and at least one quenching molecule.

14. The method of claim 12, wherein said at least one fluorophore is configured to be separated from said at least one quenching molecule by a calculated distance, wherein said calculated distance is sufficiently small so that said at least one quenching molecule quenches a fluorescent emission of said at least one fluorophore.

18. The method of claim 12, further comprising the steps of designing a probe, synthesizing said probe, and implementing said probe in said real-time PCR, wherein said designing of said probe comprises the steps of:
retrieving the DNA sequence of S. pyrogenes from a database,
finding within said DNA sequence of S. pyrogenes a first target sequence and a second target sequence, wherein said first target sequence is flanked on a first end by a DNA sequence complementary to said at least one forward primer, wherein said DNA target sequence is flanked on a second end by a DNA sequence corresponding to said at least one reverse primer, wherein said second target sequence is complementary to said first target sequence,
designing a probe comprising at least one oligonucleotide, at least one fluorophore, and at least one quenching molecule, wherein said at least one oligonucleotide is configured to hybridize with a sequence selected from the group consisting of at least one portion of the first target sequence and at least one portion of the second target sequence.
